(19) United States
(12) Patent Application Publication
(10) Pub. No.: US 2018/0265856 A1
(43) Pub. Date: Sep. 20, 2018

(54) ENDOTOXIN-REDUCED THERMOLYSIN

(71) Applicant: Amano Enzyme Inc., Nagoya-shi, Aichi (JP)

(72) Inventors: Takafumi Koyama, Kakamigahara-shi (JP); Hiroki Ido, Kakamigahara-shi (JP); Shotaro Yamaguchi, Kakamigahara-shi (JP)

(73) Assignee: Amano Enzyme Inc., Nagoya-shi, Aichi (JP)

(21) Appl. No.: 15/755,313

(22) PCT Filed: Aug. 17, 2016
(86) PCT No.: PCT/JP2016/074046

§ 371 Date: Feb. 26, 2018

(30) Foreign Application Priority Data
Aug. 28, 2015 (JP) 2015-169774

(51) Int. Cl. C12N 9/54; C12Q 1/37

(52) U.S. Cl. CPC C12N 9/54; C12Q 1/37; C12Y 304/24027

(57) ABSTRACT

It is necessary to establish a purification method effective for the removal of endotoxin, in order to obtain thermolysin containing a reduced amount of contaminant endotoxin. On the premise of the establishment, accurate measurement of the amount of endotoxin that contaminates thermolysin is required. The present invention addresses the problem of providing a novel measurement method which enables accurate measurement of the amount of endotoxin that contaminates thermolysin. The present invention also addresses the problem of providing thermolysin containing a reduced amount of contaminant thermolysin, and the use thereof. There is provided thermolysin containing contaminant endotoxin in an amount of 1 EU/mg or less. There is also provided a method for measuring endotoxin that contaminates thermolysin, the method including pretreatment of deactivating the thermolysin. An enzyme preparation including the thermolysin according to the present invention as an active ingredient has a high utility value in the field of regenerative medicine.

### TECHNICAL FIELD

The present invention relates to thermolysin. Specifically, the invention relates to thermolysin containing a reduced amount of contaminant endotoxin, a method for measuring endotoxin useful in the production thereof, and the like. The present application claims a priority based on Japanese Patent Application No. 2015-169774 filed on Aug. 28, 2015, and the entire contents of the patent application are incorporated herein by reference.

### BACKGROUND ART

Thermolysin (EC 3.4.24.27) is an enzyme classified in metal proteases, and catalyzes hydrolysis reactions using a protein as a substrate. Thermolysin is isolated, for example, from Bacillus thermoproteolyticus (for example, see Patent Literature 1 and Non-Patent Literature 1) or Geobacillus stearothermophilus (for example, strain deposited under No. NBRC12550, No. NBRC12983, No. NBRC13737 or No. NBRC100862). Also, there is a commercial thermolysin (for example, thermolysin provided by Amano Enzyme Inc.), which is commercially available.

The utilization/application of thermolysin in/to regenerative medicine is being studied as a new attempt. The regenerative medicine use requires high safety, and thus the amount of contaminant endotoxin becomes a problem.

### CITATION LIST

### Patent Literature

Patent Literature 1: JP H03-232494 A

### Non Patent Literature

Non-Patent Literature 1: Shigetoshi Endo, Journal of Hakkokogaku, 40 (1962) 346-353

### SUMMARY OF INVENTION

### Technical Problem

It is necessary to establish a purification method effective for the removal of endotoxin, in order to obtain thermolysin containing a reduced amount of contaminant endotoxin. On the premise of the establishment, accurate measurement of the amount of endotoxin that contaminates thermolysin is required. The method for measuring endotoxin, which is currently available, is substantially limited to a method of utilizing color development reactions of horseshoe crabs (for example, Limulus Color KY Test Wako provided by Wako Pure Chemical Industries, Ltd.). Since the activity of thermolysin affects the measurement values in this measurement method, the amount of endotoxin that contaminates thermolysin cannot accurately be measured. Thus, the present invention addresses the problem of providing a novel measurement method which enables the accurate measurement of the amount of endotoxin that contaminates thermolysin. Also, the present invention addresses the problem of providing thermolysin containing a reduced amount of contaminant endotoxin, and the use thereof.

### Solution to Problem

During the studies to solve the above problems, the present inventors considered that the pretreatment of a sample is important to suppress the influences of the activity of thermolysin on the measurement values. Thermolysin was not easy to deactivate without any influence on endotoxin due to its high thermal stability, but, as a result of earnest studies focusing on the thermolysin concentration in addition to the temperature condition, the present inventors succeeded in establishing a novel measurement method which is easy to operate and is remarkably excellent as compared with the conventional measurement method. When the amount of endotoxin contained in a currently available thermolysin was measured by the novel measurement method, contaminant endotoxin was detected. However, no contaminant endotoxin was detected in the thermolysin after purification using endotoxin-free instruments, facilities and materials (detection limit (0.001 EU/mg) or less). Specifically, in addition to the establishment of the novel endotoxin measurement method, the inventors succeeded in obtaining thermolysin containing an extremely reduced amount of contaminant endotoxin.

Based on the above achievement results, the present application provides the following inventions.

[1] Thermolysin containing contaminant endotoxin in an amount of 1 EU/mg or less.

[2] The thermolysin according to [1], wherein the amount of the contaminant endotoxin is 0.5 EU/mg or less.

[3] An enzyme preparation including the thermolysin according to [1] or [2] as an active ingredient.

[4] The enzyme preparation according to [3], which is used in the dispersion or recovery of cultured cells.

[5] The enzyme preparation according to [3], which is intended for regenerative medicine.

[6] The enzyme preparation according to [5], which is used in the regeneration of biotissues.

[7] A method for measuring endotoxin that contaminates thermolysin, the method including the step of deactivating the thermolysin.

[8] The measurement method according to [7], wherein the step involves heat treatment at 99° C. to 100° C. for 3 minutes to 5 minutes.

[9] The measurement method according to [8], wherein the step is carried out using a sample including the thermolysin in an amount adjusted to 180 PU/mL or less.

[10] A method for producing thermolysin, including purification in an endotoxin-free environment.

[11] The production method according to [10], including the step of confirming that the amount of contaminant endotoxin is 1 EU/mg or less by the measurement method according to any one of [7] to [9].

### BRIEF DESCRIPTION OF DRAWINGS

FIG. 1 shows studies on the conditions for heat treatment of endotoxin.

FIG. 2 shows studies on the conditions for deactivating thermolysin.

### DESCRIPTION OF EMBODIMENTS

A first aspect of the present invention relates to thermolysin containing a reduced amount of contaminant endotoxin. The thermolysin according to the present invention is characterized by containing contaminant endotoxin in an amount of 1 EU/mg or less. In this manner, in the thermolysin according to the present invention, the amount of the contaminant endotoxin is defined by a clear numerical value. Such definition has been made possible by virtue of success in development of a novel measurement method. That is, the provision of thermolysin containing a reduced amount of contaminant endotoxin could be realized by virtue of the achievement result, i.e., the development of a novel measurement method.

Preferably, the amount of the contaminant endotoxin in the thermolysin according to the present invention is 0.5 EU/mg or less. More preferably, the amount of the contaminant endotoxin in the thermolysin according to the present invention is 0.1 EU/mg or less. Still more preferably, the amount of the contaminant endotoxin in the thermolysin according to the present invention is 0.01 EU/mg or less. Most preferably, the amount of the contaminant endotoxin in the thermolysin according to the present invention is the detection limit or less. The amount of the contaminant endotoxin is calculated using the novel measurement method developed by the present inventors. The detection limit in the measurement method is 0.001 EU/mg. The thermolysin according to the present invention is preferably produced by a production method which will be described later.

Currently, several thermolysins (for example, CELASE™ provided by Roche and CIzyme™ Thermolysin provided by VitaCyte) are commercially available. The amount of contaminant endotoxin contained in CELASE™ is 3 EU/mg (COA (Certificate of Analysis) value), and that contained in CIzyme™ Thermolysin is 6.44 EU/mg (COA value).

The thermolysin specified in terms of the amount of contaminant endotoxin can be utilized as the active ingredient of preparations suitable for various uses (for example, medical uses) in which the influences of endotoxin become a problem. Therefore, the present invention also provides an enzyme preparation including the thermolysin according to the present invention as an active ingredient. The enzyme preparation according to the present invention is used, for example, in the exfoliation of cultured cells from a culturing surface, dispersion of cells (for example, separation of a cell mass into individual cells, conversion of a cell mass to smaller cell masses, or prevention of aggregation of cells), and recovery (for example, exfoliation from a culturing surface). For example, the treatment can be performed as a part of the step of preparing an implant material for regenerative medicine. Examples of other uses of the enzyme preparation according to the present invention can include the regeneration of biotissues. In this use, the enzyme preparation according to the present invention is applied to a site (i.e., an affected area) of a living body in need of tissue regeneration, alone or together with other materials (such as drugs and excipients). The enzyme preparation according to the present invention includes, as an active ingredient, thermolysin containing a reduced amount of contaminant endotoxin, and thus is suitable for utilization in the field of regenerative medicine as mentioned above. In other words, the enzyme preparation according to the present invention is characterized by having a reduced content of endotoxin, and thus has a high utility value, especially, in the field of regenerative medicine.

A second aspect of the present invention relates to a method for measuring endotoxin that contaminates thermolysin. The measurement method according to the present invention has the most characteristic feature of including the step of deactivating the thermolysin. The step of deactivating the thermolysin (thermolysin deactivation step) is carried out prior to the detection or measurement of endotoxin in a sample. In brief, the present invention involves carrying out the thermolysin deactivation step as pretreatment. In the thermolysin deactivation step, the sample is heat-treated under predetermined conditions to deactivate the thermolysin in the sample while suppressing the influences on endotoxin, thereby preventing the thermolysin in the sample from affecting the detection value/measurement value during the detection/measurement of the endotoxin. While the heat treatment conditions for the thermolysin deactivation step are not particularly limited so long as this object is attained, the sample is preferably treated at 99 to 100° C. for 3 to 5 minutes. For example, a container in which the sample is housed is maintained in boiling water, thereby making it possible to realize the treatment. While the pH of the sample is not particularly limited, a sample having a pH adjusted to pH 6 to 7 is used to carry out the thermolysin deactivation step.

The thermolysin deactivation step is in danger of deactivating a part of endotoxin in the sample. However, since high correlation is observed between the treatment conditions and the deactivation amount, the amount of endotoxin in consideration of the deactivation amount can be calculated by multiplying the measurement value by a correction coefficient. The correction coefficient can be obtained from the deactivation amount when a sample containing endotoxin alone is treated under the treatment conditions employed.

As a result of the studies made by the present inventors, it has been revealed that the concentration of the thermolysin in the sample is also one of important elements in obtaining accurate measurement results. Thus, a sample including thermolysin in an amount adjusted to 180 PU/mL or less (i.e., 0 PU/mL to 180 PU/mL) is used to carry out the thermolysin deactivation step in a preferred embodiment. Meanwhile, the activity value of thermolysin is calculated, as the activity in the casein decomposition method (the amount of an enzyme liberating 1 μg of tyrosine for 1 minute is defined as 1 PU (Protease Unit)), using a standard product (7,000,000 PU/g of thermolysin (manufactured by Amano Enzyme Inc.)).

As is apparent from the above description, the measurement method according to the present invention is characterized by the pretreatment (thermolysin deactivation step). After the pretreatment, endotoxin is detected or measured by a known endotoxin test (so-called, Limulus method). As for the details of the method for conducting the endotoxin test, see the 15th Revised Japanese Pharmacopoeia, 4.01 Endotoxin Test Method (2008) and FDA guideline “Guideline on Validation of the Limulus Amebocyte Lysate Test as an End-Product Endotoxin Test for Human and Animal Parenteral Drugs, Biological Products, and Medical Devices” (1987). Several kits for the Limulus method are commercially available (for example, Limulus Color KY Test Wako provided by Wako Pure Chemical Industries, Ltd.). The measurement method according to the present invention can be carried out more conveniently by utilizing such a commercially available kit.

A further aspect of the present invention relates to a method for producing thermolysin. The production method of the present invention involves purifying the provided thermolysin under specific conditions to reduce endotoxin. The starting raw material, thermolysin, can be obtained by isolation, for example, from Bacillus thermoproteolyticus (see Shigetoshi Endo, Journal of Hakkokogaku, 40 (1962) 346-353 and JP H03-232494 A). As the starting raw material, thermolysin derived from Geobacillus stearothermophilus (for example, strain deposited under No. NBRC12550, No. NBRC12983, No. NBRC13737 or No. NBRC100862) may be used. On the other hand, a commercially available thermolysin (for example, thermolysin provided by Amano Enzyme Inc.) may be used as the starting raw material.

The production method of the present invention is characterized by performing purification in an endotoxin-free environment. The “endotoxin-free environment” refers to conditions in which instruments, facilities and materials which are free from inclusion of, attachment of, and contamination with, endotoxin are used. For example, sterile water from which endotoxin has been removed, for example, by filter treatment is used as water for dilution, washing and the like. In general, water of injection grade corresponds to the sterile water. Filtration, centrifugation, dilution, concentration, salting-out, dialysis, dissolution, adsorption and elution, drying, and the like can be exemplified as the purifying operations. Preferably, it is confirmed that the amount of the contaminant endotoxin is 1 EU/mg or less after the purification step. This confirmation step can be carried out using the measurement method according to the present invention.

### EXAMPLES

### 1. Studies on Conditions for Heat Treatment of Standard Endotoxin Product

### (1) Method

A standard endotoxin product (Control Standard Endotoxin) attached to Limulus Color KY Test Wako (Wako Pure Chemical Industries, Ltd.) was diluted to concentrations (0.001 to 1 EU/mL). The diluted products were heated in boiling water (boiling water bath) for a defined time (0 to 10 minute(s)). The heated products were immediately cooled, and the amount of endotoxin was measured by Limulus Color KY Test Wako to calculate relative values when the measurement values obtained under the condition: a heating time of 0 minute in the respective concentrations were defined as 100%.

### (2) Results

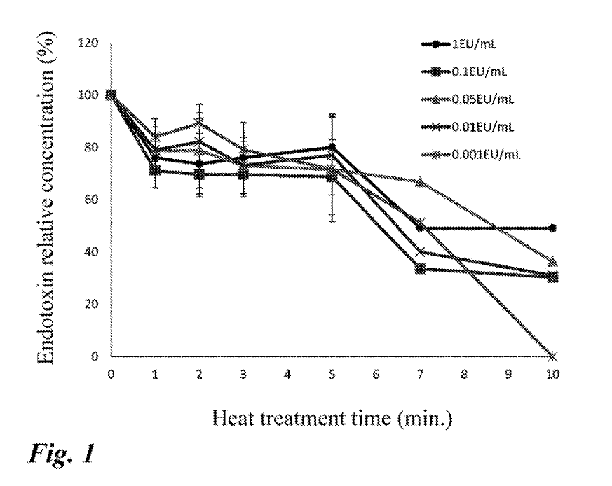

The measurement results are shown in FIG. 1. The treatment time ranging from 1 minute to 5 minutes did not affect the endotoxin concentration. Thus, the condition for heat treatment by boiling water bath was determined to be suitably 1 minute to 5 minutes.

### 2. Studies on Conditions for Deactivating Thermolysin

### (1) Method

Thermolysin solutions having predetermined concentrations (90, 180 and 900 PU/mL) (pH of 6 to 7) were provided, and subjected to pretreatment at a predetermined temperature (70° C., 80° C. or 90° C. or in boiling bath) for a predetermined time (1, 2, 3 or 5 minute(s)). After the pretreatment, the hydrolysis of furyl acryloyl-glycyl-L-leucine-amide (FAGLA) was measured to evaluate the enzyme activity. A specimen inspection system TBA-120FR (Toshiba Medical Systems Corporation) was used for the measurement of the hydrolysis. The measurement values of the respective samples were calculated as compared with the absorbance of 7,000,000 PU/g of thermolysin (manufactured by Amano Enzyme Inc.). The measurement values of the respective samples were compared with the measurement values of the samples without pretreatment to calculate the relative activity values.

### (2) Results

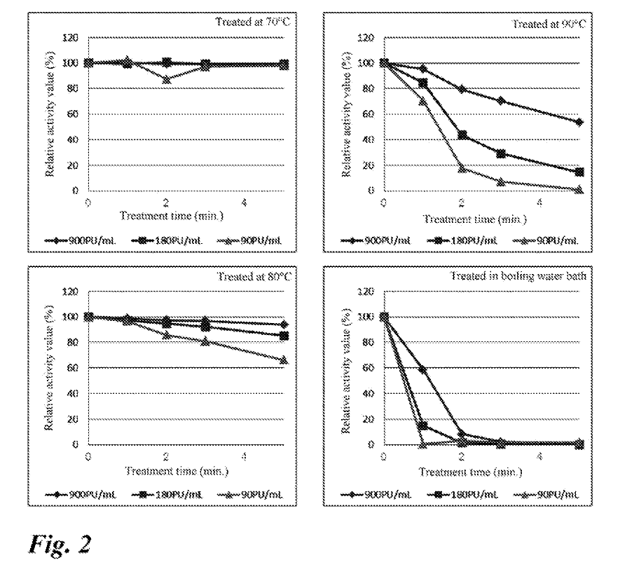

It was confirmed whether the deactivation of thermolysin was observed upon heating treatment for a time within 5 minutes which did not affect the measurement of endotoxin. The deactivation of thermolysin is observed by heating in a boiling water bath for 3 to 5 minutes while the enzyme activity is adjusted to 180 PU/mL or less (FIG. 2), and thermolysin suitable for the measurement of endotoxin is obtained.

### 3. Purification of Thermolysin

Thermolysin (manufactured by Amano Enzyme Inc.) was purified using endotoxin-free instruments, facilities and materials. The samples after the purification were diluted so that the enzyme activity was 180 PU/mL or less, and then the diluted samples were subjected to pretreatment (at 99 to 100° C. for 3 minutes). The samples after the pretreatment were immediately moved onto ice, and rapidly cooled. Thereafter, the amount of endotoxin was measured by Limulus Color KY Test Wako. As a result of the measurement, the amount of endotoxin was the detection limit (0.001 EU/mg) or less (N=3).

### INDUSTRIAL APPLICABILITY

The thermolysin according to the present invention contains an extremely reduced amount of contaminant endotoxin. Due to this characteristic feature, the enzyme preparation including the thermolysin according to the present invention as an active ingredient has a high utility value in the field of regenerative medicine. On the other hand, the amount of the endotoxin that contaminates thermolysin can be understood more accurately according to the measurement method of the present invention. Accordingly, the measurement method according to the present invention can be utilized to provide an enzyme preparation having high and stable quality.

The present invention is not limited in any way to the above explanation of the embodiments or the examples of the present invention. Various modifications that can be easily conceived by those skilled in the art without departing from the scope of the claims fall within the scope of the present invention. All of contents of the literatures, the patent laid-open publications, the patent gazette and the like specified in the description are incorporated herein by reference.

1. Thermolysin containing contaminant endotoxin in an amount of 1 EU/mg or less.

2. The thermolysin according to claim 1, wherein the amount of the contaminant endotoxin is 0.5 EU/mg or less.

3. An enzyme preparation comprising the thermolysin according to claim 1 as an active ingredient.

4. The enzyme preparation according to claim 3, which is used in the dispersion or recovery of cultured cells.

5. The enzyme preparation according to claim 3, which is intended for regenerative medicine.

6. The enzyme preparation according to claim 5, which is used in the regeneration of biotissues.

7. A method for measuring endotoxin that contaminates thermolysin, the method comprising the step of deactivating the thermolysin.

8. The measurement method according to claim 7, wherein the step involves heat treatment at 99° C. to 100° C. for 3 minutes to 5 minutes.

9. The measurement method according to claim 8, wherein the step is carried out using a sample comprising the thermolysin in an amount adjusted to 180 PU/mL or less.

10. A method for producing thermolysin, comprising purification in an endotoxin-free environment.

11. The production method according to claim 10, comprising the step of confirming that the amount of contaminant endotoxin is 1 EU/mg or less by the measurement method according to claim 7.
